SEQUENCE LISTING (2 sequences)

SEQ ID NO: 1 - RNA, 21 nt, Artificial Sequence
  siRNA of 21-mers corresponding to mouse PLD1
aacacguuagcuaagugguau

SEQ ID NO: 2 - RNA, 21 nt, Artificial Sequence
  siRNA of 21-mers corresponding to mouse PLD2
aacuccauccaggcuauucug

(19) United States
(12) Patent Application Publication
(10) Pub. No.: US 2009/0312250 A1
(43) Pub. Date: Dec. 17, 2009

(54) EPIDERMAL GROWTH FACTOR INCREASING INSULIN SECRETION AND LOWERING BLOOD GLUCOSE

(75) Inventors: Sung-Ho Ryu, Kyungsangbuk-do (KR); Hye-Young Lee, Kyungsangbuk-do (KR); Kyung-Moo Yea, Kyungsangbuk-do (KR); Byoung-Dae Lee, Busan-city (KR); Young-Chan Chae, Kyungsangbuk-do (KR); Hyeon-Soo Kim, Kyungsangbuk-do (KR); Seon-Hee Kim, Kyungsangbuk-do (KR); Pann-Ghill Suh, Kyungsangbuk-do (KR)

Correspondence Address: SHERIDAN ROSS PC, 1560 BROADWAY, SUITE 1200, DENVER, CO, 80202, US

(73) Assignee: POSTECH ACADEMY-INDUSTRY FOUNDATION, Pohang-city (KR)

(21) Appl. No.: 12/373,691

(22) PCT Filed: Jul. 16, 2007
(86) PCT No.: PCT/KR2007/003451

§ 371 Date: Jan. 13, 2009

Related U.S. Application Data
(60) Provisional application No. 60/807,374, filed on Jul. 14, 2006.

(51) Int. Cl. A61K 38/18

(52) U.S. Cl. 514/12

(57) ABSTRACT

The present inventors show that a brief exposure to EGF stimulates insulin secretion glucose-independently via a Ca2+ influx- and PLD2-dependent mechanism. Furthermore, the present invention shows that EGF is a novel secretagogue that lowers plasma glucose levels in normal and diabetic mice, suggesting the potential for EGF treatment in diabetes.

### CROSS REFERENCE TO RELATED APPLICATION

This application claims priority to and the benefit of U.S. provisional application No. 60/807,374 filed in the United State of Patent and Trademark Office on Jul. 14, 2006, the entire content of which is incorporated hereinto by reference.

### FIELD OF INVENTION

The present invention relate to an agent of preventing or treating a diabetes mellitus and a method of preventing or treating a diabetes mellitus. In addition, the present invention is directed to an agent of controlling blood glucose level and a method of controlling blood glucose level, a method of identifying an agent that induces glucose-independent insulin secretion in a mammal, and a method of diagnosing a diabetes mellitus or low blood glucose level.

### BACKGROUND OF THE INVENTION

The main function of pancreatic β-cells is to synthesize and secrete insulin at appropriate rates to limit blood glucose fluctuations. Excessive secretion of insulin causes hypoglycemia, and insufficient secretion leads to diabetes. It is therefore not surprising that insulin secretion is subject to very tight control to ensure glucose homeostasis in the body. Insulin is stored in secretory granules in pancreatic β-cells and, upon stimulation with secretagogues, is released by exocytosis. The level of β-cell activity is determined by several different stimulators, including glucose, amino acids, fatty acids, neurotransmitters, and hormones. In spite of intensive studies, the processes that are involved in this stimulus-secretion coupling and that maintain exquisite control of insulin release are still incompletely understood.

Diabetes is one of the most common endocrine diseases across all age groups and populations. There are two major forms of diabetes mellitus: insulin-dependent (Type 1) diabetes mellitus (IDDM) which accounts for 5 to 10% of all cases, and non-insulin-dependent (Type 2) diabetes mellitus (NIDDM) which comprises roughly 90% of cases. Type 2 diabetes is associated with increasing age however there is a trend of increasing numbers of young people diagnosed with NIDDM, so-called maturity onset diabetes of the young (MODY). In both Type 1 and Type 2 cases, there is a loss of insulin secretion, either through destruction of the β-cells in the pancreas or defective secretion or production of insulin. In NIDDM, patients typically begin therapy by following a regimen of an optimal diet, weight reduction and exercise.

Type 2 diabetes mellitus is characterized by both insulin resistance and impaired insulin secretion. The control of insulin secretion is primarily regulated by glucose itself, but also involves an array of metabolic, neural, hormonal, and sometimes pharmacological factors. Initiators can increase insulin secretion in the absence of other stimulation, but potentiators require the presence of an initiator, usually glucose (Hedeskov C J et al., Physiol Rev 60:442-509, 1980). Many reports have suggested that hypoglycemia caused by diabetes treatment poses a serious problem.

Epidermal growth factor (EGF) is an important growth factor for the proliferation of different types of cells, especially fibroblasts and epithelial cells. EGF can also induce secretion events, including acrosomal exocytosis and the secretion of several hormones. Some members of the EGF family are proposed to have a role in the development of the pancreas. EGF and leukemia inhibitory factor (LIF) treatment in vitro generated an insulin-producing β-cell mass (Baeyens L et al., Diabetologia 48:49-57, 2005). The EGFR is expressed throughout the human fetal pancreas, and mice lacking EGFR show abnormal pancreatic islets (Miettinen P J, et al., Development 127:2617-2627, 2000). EGF also has shown to be related in the insulin content of rat pancreatic β-cells and regeneration of them (Li L, et al., Diabetes 53:608-615, 2004; Brand S J, et al., Pharmacol Toxicol 91:414-420, 2002; Suarez-Pinzon W L et al., Diabetes 54:2596-2601, 2005). EGF is also produced in the pancreas and its circulating levels and the EGFR are reduced in diabetic animals (Burgess A W, Br Med Bull 45:401-424, 1989; Kasayama S, et al., Proc Natl Acad Sci USA 86:7644-7648, 1989; Kashimata M, et al., Biochim Biophys Acta 923:496-500, 1987). However, the role of EGF in glucose regulation by modulating pancreatic function such as insulin secretion has not been studied yet.

Insulin secretion is mainly triggered by the elevation of intracellular Ca²⁺ but it can be modulated by several cellular signals such as protein kinases and phospholipases. Of them, mammalian phospholipase D (PLD) is a membrane bound enzyme that hydrolyzes phosphatidyl choline (PC) to generate a multifunctional lipid, phosphatidic acid (PA), in response to a variety of signals, including growth factors (Exton J H, Biochim Biophys Acta 1439:121-133, 1999). PA is an intracellular lipid second messenger involved in multiple physiological events. These findings suggest that agonist-induced PLD activation may play roles in multiple signaling events (Jones D, et al., Biochim Biophys Acta 1439:229-244, 1999; Honda A, et al., Cell 99:521-532, 1999). To date, two types of mammalian PLD, PLD1 and PLD2, have been cloned. They share a sequence homology of around 50% and contain similar regulatory domains, but show differences in localization and regulatory protein interactions (Frohman M A, et al., Biochim Biophys Acta 1439:175-186, 1999). PLD activity may be involved in various trafficking processes, particularly in the regulation of exocytosis (Jones D, et al., Biochim Biophys Acta 1439:229-244, 1999). PLD1 and PLD2 regulate different phases of exocytosis in mast cells by a two-step process (Choi W S, et al., J Immunol 168:5682-5689, 2002). In addition, PA is an important mediator of insulin exocytosis (Metz S A, Biochem J 270:427-435, 1990).

### SUMMARY OF THE INVENTION

However, the role of EGF in glucose regulation by modulating pancreatic function such as insulin secretion has not been studied yet. The specific regulation of PLDs by secretagogues remains unclear.

The present inventors show that a brief exposure to EGF stimulates insulin secretion glucose-independently via a Ca2+ influx- and PLD2-dependent mechanism. Furthermore, the present invention shows that EGF is a novel secretagogue that lowers plasma glucose levels in normal and diabetic mice, suggesting the potential for EGF treatment in diabetes.

In one embodiment of the present invention, a pharmaceutical composition for preventing or treating diabetes mellitus comprising EGF as an effective agent is provided. The present invention provides an insulin-secreting agent comprising EGF, more preferably a glucose-independent insulin-secreting agent comprising EGF and wherein the EGF stimulates the insulin secretion of the pancreatic beta-cell in a glucose-independent manner. The present invention also provides a method of treating diabetes mellitus comprising administering to a subject an effective amount of EGF, wherein the amount of the EGF initiates the insulin secretion and low

In another embodiment, the present invention provides an agent of controlling a blood glucose level comprising EGF, and a method of controlling blood glucose level comprising administering an effective amount of a EGF to the mammal in need thereof.

In further embodiment, the present invention provides a diagnosing kit of the diabetes mellitus comprising the EGF and a method of diagnosing diabetes mellitus in a mammal using the EGF. More specifically, the method of diagnosing the diabetes mellitus can be performed by preparing blood sample of a subject, and determining the EGF concentration in the blood sample with antigen-antibody reaction.

In additional embodiment, the present invention provides a method of identifying an agent that induces glucose-independent insulin secretion in a mammal, the method comprising using the EGF.

### BRIEF DESCRIPTION OF THE DRAWING

A more complete appreciation of the invention, and many of the attendant advantages thereof, will be readily apparent as the same becomes better understood by reference to the following detailed description when considered in conjunction with the accompanying drawing, wherein:

FIG. 1A to 1C show that EGF rapidly and glucose-independently stimulates insulin secretion in MIN6 cells. EGF showed time- and dose-dependent stimulation of insulin secretion from MIN6 cells, with kinetics more rapid than glucose (FIGS. 1A and 1B), and EGF increased insulin levels at basal concentration of glucose (2.7 mM), and additively increased glucose-induced insulin release at high (11 mM) glucose levels as well (FIG. 1C).

FIG. 2A to 2B show that Ca²⁺ influx mediates the EGF-triggered insulin secretion in MIN6 cells. FIG. 2A demonstrated that EGF stimulated extracellular Ca²⁺ influx, which could be reduced by EGTA treatment, and EGF-induced insulin secretion from MIN6 cells was reduced by Ca²⁺ chelators (FIG. 2B).

FIG. 3C to 3D show that PLD2 specifically involves in the EGF-dependent insulin secretion. PLD was activated rapidly (within 2 min) by EGF stimulation (FIG. 3A), EGF-dependent insulin secretion was inhibited by 1-butanol, a PLD inhibitor, treatment, but not by t-butanol treatment as a control (FIG. 3B), PLD2 exclusively mediated EGF-dependent insulin secretion and overexpression of PLD1 showed a limited effect (upper panel of FIG. 3C), silencing of PLD2 abolished EGF-induced insulin secretion (upper panel of FIG. 3D), and EGF-dependent PLD activity as measured with PBt formation was modulated exclusively by PLD2 overexpression or silencing (lower panels of FIGS. 3C and 3D).

FIG. 4A to 4B show that Ca²⁺ influx is critical for the EGF-induced PLD activation, Blocking Ca²⁺ influx by using EGTA or BAPTA/AM inhibited most of the PLD activity (FIG. 4A), inhibiting PLD activity by silencing PLD isozymes, which the successful silencing of PLDs was confirmed by western blotting, had little effect on EGF-dependent Ca²⁺ influx (FIG. 4B)

FIG. 5A to 5C show that Insulin secretion is increased by EGF in mouse pancreatic islets through Ca²⁺ influx and PLD activity, EGF rapidly increased insulin secretion (FIG. 5A), Inhibiting Ca²⁺ influx or PLD activity completely blocked the EGF-induced insulin secretion (FIGS. 5B and 5C).

FIG. 6A to 6E show that EGF lowers plasma glucose and increases plasma insulin levels, glucose-lowering effect of EGF (50 g/kg) had a similar potency to insulin and had a dose-dependency (FIG. 6A). Moreover, EGF and glucose (0.5 g/kg) also both increased plasma insulin levels (FIG. 6B), oral injection of glucose caused the elevation of plasma EGF levels comparing with saline treatment (FIG. 6C), EGF also reduced plasma glucose in obese db/db mice and increased plasma insulin levels (FIGS. 6D and 6E)

### DETAILED DESCRIPTION

A more complete appreciation of the invention, and many of the attendant advantages thereof, will be readily apparent as the same becomes better understood by reference to the following detailed description.

Epidermal growth factor (EGF) is synthesized in the pancreas and diabetic animals have low levels of EGF. However, the role of EGF in regulating the major function of pancreas such as glucose homeostasis has not been studied. Here, the present invention shows that EGF rapidly increased insulin secretion in mouse pancreatic islets, as well as in a pancreatic-cell line. These events were dependent on Ca²⁺ influx and PLD activity, particularly PLD2, as determined using pharmacological blockers and molecular manipulations such as overexpression and siRNA of PLD isozymes. In addition, EGF also increased plasma insulin levels and mediated glucose lowering in normal and diabetic mice. Here, for the first time, the present invention provides evidences that EGF is a novel secretagogue regulating plasma glucose levels and an agent for preventing or treating diabetes mellitus.

In an embodiment of the present invention, the present invention is directed to a pharmaceutical composition for preventing or treating diabetes mellitus comprising EGF and a pharmaceutically acceptable carrier. More specifically, the EGF can be human EGF. The EGF stimulates the insulin secretion from pancreatic beta-cell in a glucose-independent manner. The EGF simulates the insulin secretion through Ca2+ influx and PLP2 activation in pancreatic beta-cells or pancreatic islets.

The EGF is administered in an amount of 5 μg/kg to 100 μg/kg by weight of the subject, and more preferably, 10 μg/kg to 60 μg/kg by weight of the subject.

In another embodiment, the present invention provides a method for controlling blood glucose levels in a subject in need thereof comprising administering an effective amount of EGF to the subject.

In another embodiment, the present invention provides a method for controlling blood insulin levels in a subject in need thereof comprising administering an effective amount of EGF to the subject.

In the method of controlling blood glucose levels in a subject or controlling blood insulin levels in a subject, the controlling blood glucose level is performed by regulating the blood insulin levels in a glucose-independent manner. The EGF can be human EGF. The effective amount is 5 μg/kg to 100 μg/kg by weight of the subject, and more preferably, 10 μg/kg to 60 μg/kg by weight of the subject. The EGF is administered orally, subcutaneously, intravenously, or intramuscularly. The subject is a patient suffering from diabetes mellitus or a normal subject.

Dosage forms of a pharmaceutical composition of the present invention or its respective active ingredients include oral dosage forms such as tablets, capsules (including soft capsules and microcapsules), powders, granules, syrups, and etc.; and non-oral dosage forms such as injections (e.g., subcutaneous injections, intravenous injections, intramuscular injections, intraperitoneal injections, etc.), external application forms (e.g., nasal spray preparations, transdermal preparations, ointments, etc.), suppositories (e.g., rectal suppositories, vaginal suppositories, etc.), pellets, drip infusions, and etc.

The dosage of a pharmaceutical composition of the present invention may be appropriately determined with reference to the dosage recommended for the respective drug(s), and can be selected appropriately according to the subject, the age and body weight of the subject, current clinical status, administration time, dosage form, method of administration, combination of the drug(s), and etc. The dosage of an insulin sensitizer and an anorectic can be selected appropriately based on clinically used dosage. For administration of an insulin sensitizer to an adult diabetic patient (body weight: 50 kg), for instance, the dose per day is usually 0.01 to 1000 mg, preferably 0.1 to 500 mg. This dose can be administered once to several times a day.

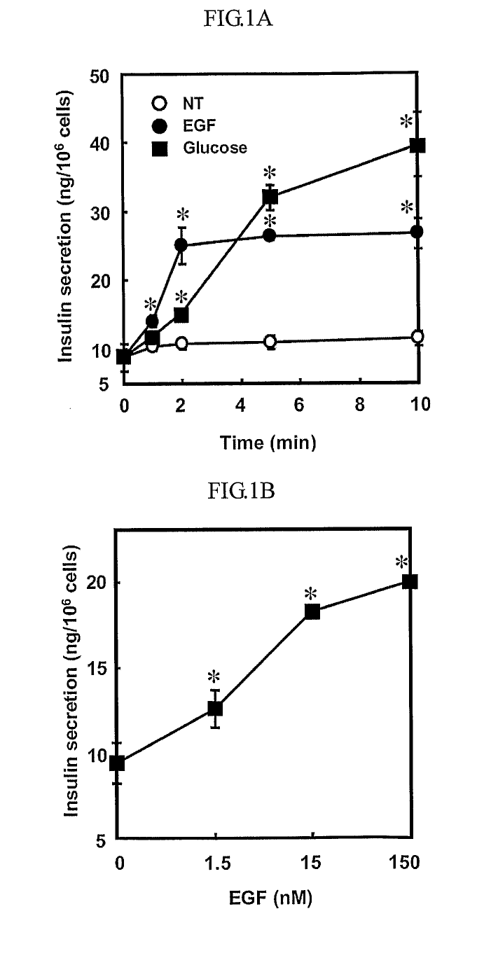

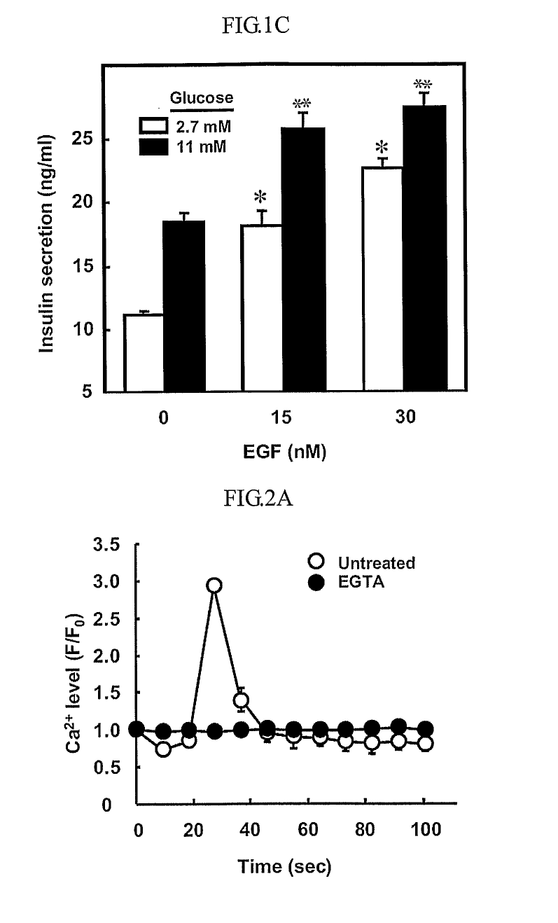

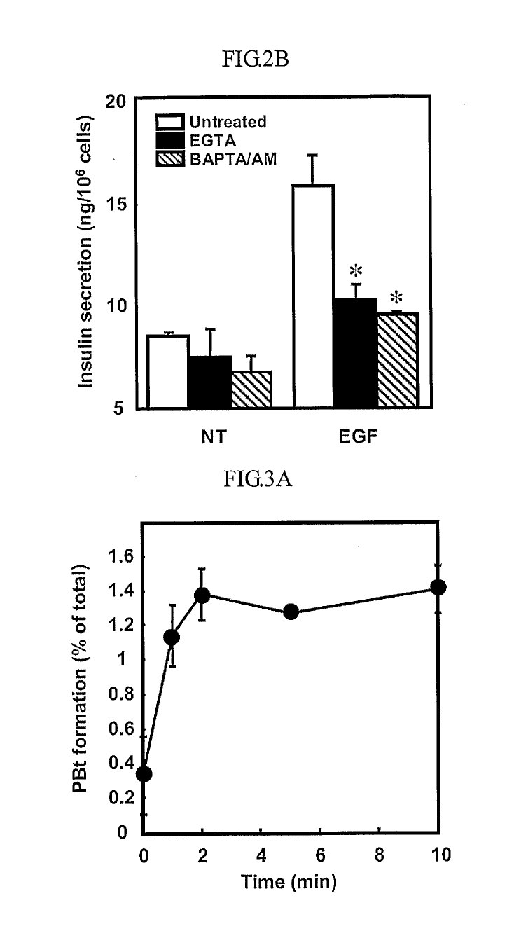

EGF requires only brief exposure (1 min) to stimulate insulin secretion (FIG. 1A), and increases Ca²⁺ levels when treated alone (FIG. 2A), indicating that it can function as an initiator. Furthermore, EGF additively stimulates glucose-dependent insulin secretion (FIG. 1C), which means that EGF effect is glucose-independent. Insulin secretion by glucose has a biphasic pattern, with a peak around 5 min, a nadir at 10 min, and a slowly increasing time course thereafter, and this first phase is key for the insulin-dependent processes that ensure glucose homeostasis (Caumo A, et al., Am J Physiol Endocrinol Metab 287:E371-385, 2004). The time course of EGF receptor-mediated insulin secretion is similar to neurotransmitter release in neuronal cells, and is more rapid than the first phase of glucose-dependent insulin secretion from pancreatic-cells. EGF-induced insulin release required rapid Ca²⁺ influx comparing with glucose, which requires 3-4 min for Ca²⁺ influx mainly due to the time for glucose metabolism and delayed change of ATP/ADP ratio (data not shown). Insulin secretion sometimes can be regulated through classical signaling cascades involving transmembrane receptors, heterotrimeric G-proteins, and second messengers (Rosenbaum T, et al., Diabetes 50:1755-1762, 2001; Itoh Y, et al., Nature 422:173-176, 2003; Mears D, J Membr Biol 200:57-66, 2004). Therefore, EGF receptor-mediated regulation of insulin secretion is not unreasonable. Here, a new role for EGF is defined as an initiator of insulin secretion, both in vitro and in vivo, indicating the therapeutic potential of EGF in diabetes.

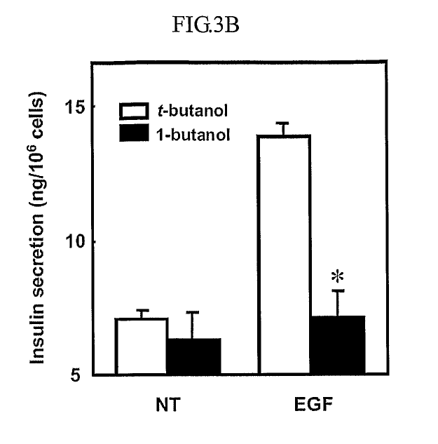

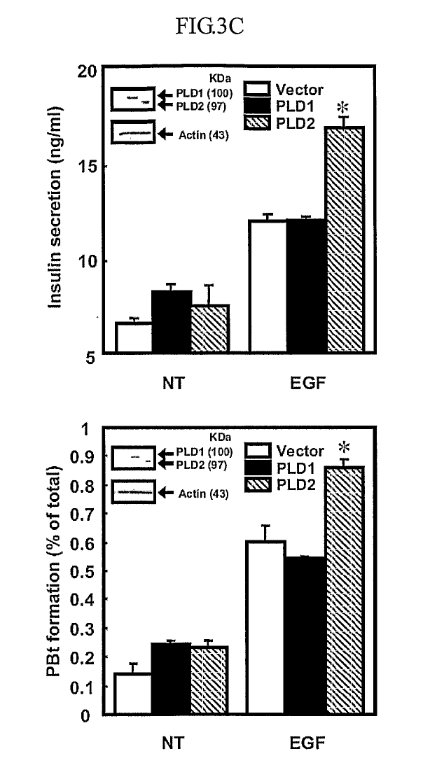

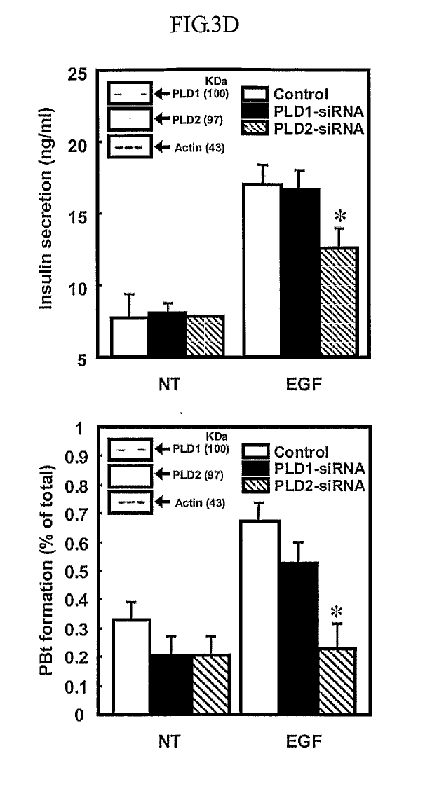

PLD is activated by EGF stimulation and this activation is a very rapid process than by other PLD-regulating molecules such as glucose etc. The mechanism underlying EGF-mediated activation of PLD remains controversial. Among them, EGF-dependent Ca²⁺ increases activate protein kinase C (PKC) and lead to PLD activation (Yeo E J, et al., J Biol Chem 270:3980-3988, 1995). Another report suggested that Ca²⁺ influx is associated with activation of PLD, and that PKC is involved in this process (Sun S H, et al., J Neurochem 73:334-343, 1999). However, there are limited studies about Ca²⁺-mediated PLD activation of specific isozymes. In the present study, the present inventors identified PLD2 as a Ca²⁺-dependent isozyme in the pancreatic β-cells by EGF treatment (FIGS. 3C and 3D). Although PLD1 and 2 share a sequence homology of around 50% and contain similar regulatory domains (Frohman M A, et al., Biochim Biophys Acta 1439:175-186, 1999), they show differences in localization and regulatory protein interactions (Min D S, et al., Mol Cells 11:369-378, 2001; Hiroyama M, et al., J Cell Biochem 95:149-164, 2005). The previous report suggested that PLD1 and PLD2 regulate different phases of exocytosis in mast cells via a two-step process (Choi W S, et al., J Immunol 168:5682-5689, 2002): translocation of granules to the cell periphery, regulated by granule-associated PLD1, and a Ca²⁺-dependent fusion of granules with the plasma membrane, regulated by plasma membrane-associated PLD2. Differently with the previous report suggesting PLD1 as a mediator of glucose-stimulated insulin secretion (Hughes W E, et al., J Biol Chem 279:27534-27541, 2004), in our hands, EGF stimulation required PLD2 activation. The specific activation of PLD1 by glucose and PLD2 by EGF has different kinetics and the mechanisms require further clarification. We detected PLD2 in MIN6 cells, both by western blotting using a PLD2-specific antibody (FIGS. 3C and 3D) and by RT-PCR (data not shown). Furthermore, the present inventors used overexpression and silencing strategies to determine that PLD2, not PLD1, mediated EGF-dependent insulin secretion (FIGS. 3C and 3D). These results postulate that glucose stimulates insulin secretion via PLD1 with a relatively late time course, whereas EGF activates plasma membrane-localized PLD2, leading to rapid fusion of predocked insulin granules with the plasma membrane. Our work supports the notion that PLD1 and PLD2 mediate different pathways for regulating insulin secretion. Since PLDs are important molecules in exocytotic processes, studying PLDs will provide significant insight into the regulatory mechanisms of insulin secretion. The different regulatory mechanisms of PLD1 and PLD2 in insulin secretion require future study.

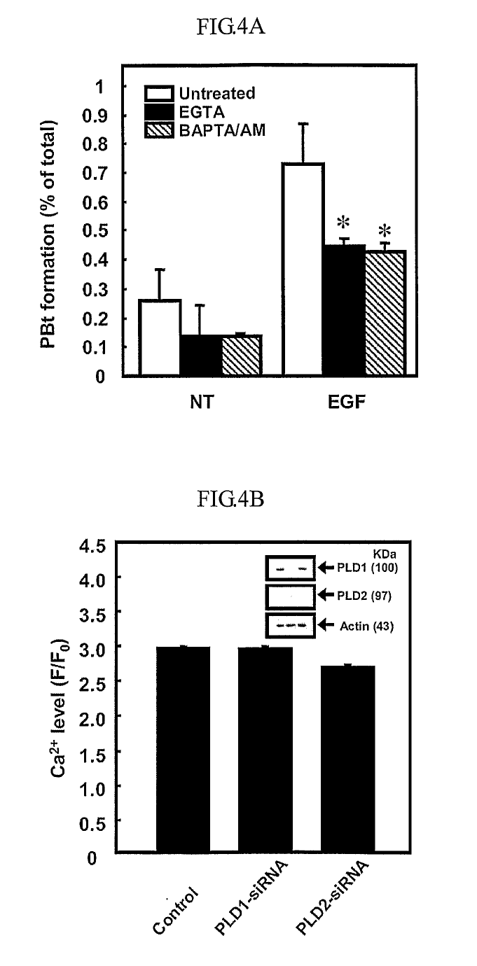

Metabolism of glucose results in closure of ATP-sensitive K⁺ channels, and the subsequent plasma membrane depolarization opens voltage-sensitive Ca²⁺ channels (Henquin J C, Diabetes 49:1751-1760, 2000). The resultant rise in the cytoplasmic free Ca²⁺ concentration is both necessary and sufficient for triggering an initial phase of insulin release that is mediated by fusion of predocked insulin granules with the plasma membrane (Mears D, J Membr Biol 200:57-66, 2004). Our results show that EGF-stimulated Ca²⁺ influx mediates insulin secretion (FIG. 2B). In the present invention, the present inventors determined that EGF-stimulated insulin secretion required both Ca²⁺ influx and PLD activity (FIGS. 2B and 3B). Our findings (FIG. 4) suggest that Ca²⁺ influx is an upstream signal for PLD activity. The present invention indicates a close relationship between secretagogue-induced Ca²⁺ influx and PLD activity in insulin secretion by pancreatic β-cells.

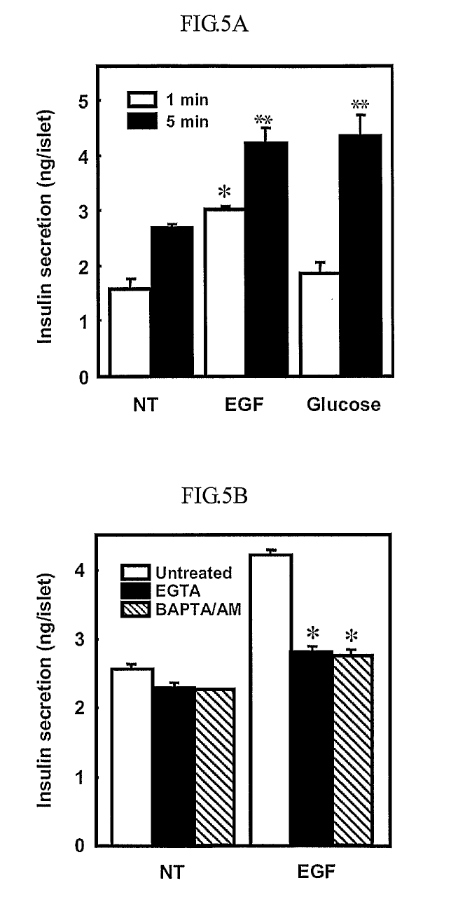

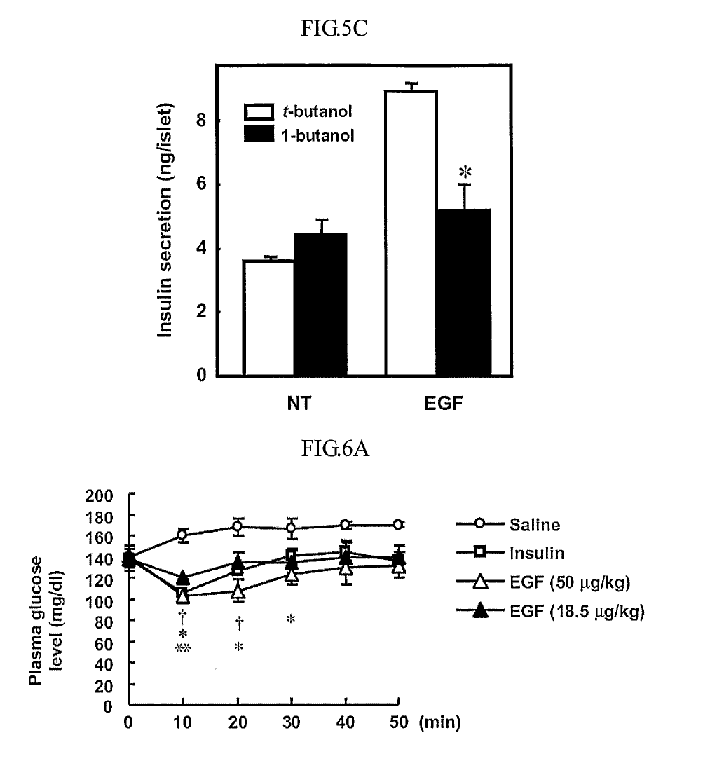

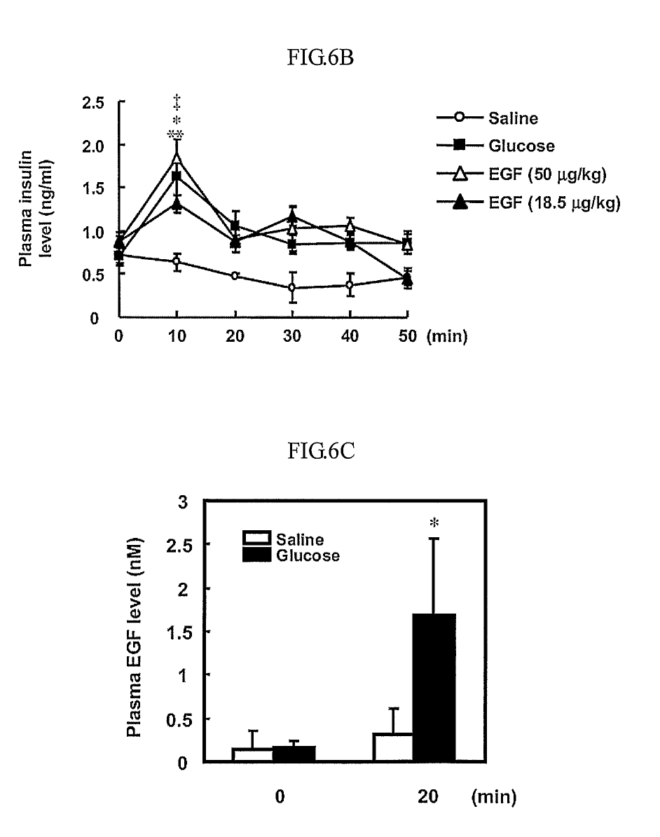

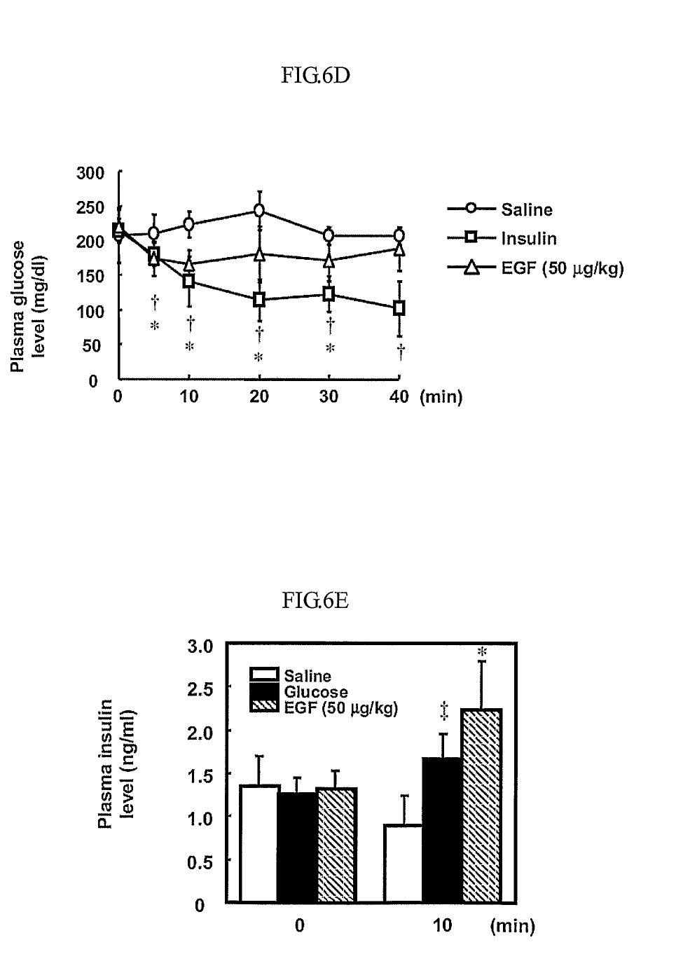

EGF regulates pancreatic function, and is produced in the pancreas and pancreatic juice (Huotari M A, et al., Endocrinology 139:1494-1499, 1998). EGFR is expressed throughout the human fetal pancreas, and mice lacking EGFR showed abnormal formation of pancreatic islets. Some members of the EGF family have a role in the development of the pancreas. EGF regulates the insulin content of rat pancreatic β-cells, as well as their regeneration. Furthermore, EGF deficiency is associated with diabetes mellitus: in diabetic animals, EGF or EGFR levels are decreased in various organs or fluids, such as liver, the submandibular gland, plasma, and milk (Thulesen J, et al., Endocr Regul 27:139-144, 1993). Interestingly, levels of these proteins often recover after insulin curative treatment, and EGF and insulin act synergistically during diabetic healing (Hennessey P J, et al., Arch Surg 125:926-929, 1990). Although EGF shows crosstalk with GLP-1-dependent signaling, which upregulates insulin secretion (MacDonald P E, et al., J Biol Chem 278:52446-52453, 2003), there has been no report showing that EGF can acutely regulate the insulin secretion. Consistent with our in vitro findings from MIN6 (FIG. 1), RINm5F (Data not shown) cell line and mouse pancreatic islets (FIG. 5) that EGF could stimulate insulin release, the present inventors found that EGF increased plasma insulin level and decreased plasma glucose level in normal and even in diabetic mice (FIG. 6). Furthermore, the present inventors observed that physiological EGF levels were elevated by glucose injection. From these results, the present inventors speculate that physiological EGF rapidly increases insulin secretion, and this process might be important in short-term regulation of plasma glucose levels. It is likely that EGF-dependent insulin secretion plays a similar function as glucose on glucose homeostasis in our body. Reducing the endogenous level of EGF using knock down, antibody, or aptamer would indicate the physiological function of EGF on glucose and insulin homeostasis. Taken together the role of EGF on insulin secretion as well as β-cell regeneration, these observations may contribute to a better understanding of the pathophysiology of diabetes mellitus, where serum EGF levels are diminished. Furthermore, the effect of EGF in diabetic mice indicates that the usefulness of EGF as a potential therapeutics of diabetes.

The present invention is further explained in more detail with reference to the following examples. These examples, however, should not be interpreted as limiting the scope of the present invention in any manner.

Materials

The enhanced chemiluminescence (ECL) kit was purchased from Amersham Pharmacia Biotech (Buckinghamshire, United Kingdom); [³H]myristic acid from Dupont NEN (Boston, Mass.); Silica Gel 60 thin-layer chromatography plates from MERCK (Darmstadt, Germany); Dulbecco's modified Eagle's medium (DMEM), RPMI 1640 and LipofectAMINE from Invitrogen (Carlsbad, Calif.); Fetal calf serum from HyClone (Logan, Utah); EGF from the Daewoong Pharmaceutical Company (Seoul, Republic of Korea); Horseradish peroxidase-conjugated goat anti-rabbit IgG and anti-mouse IgA, IgG, and IgM from Kirkegaard and Perry Laboratories, Inc. (Gaithersburg, Md.); and Fluo-3 AM from Molecular Probes (Eugene, Oreg.). A polyclonal antibody (mSTP4) recognizing both PLD1 and PLD2 was produced as described previously (Lee et al., Biochim Biophys Acta 1347:199-204, 1997). A PLD2-specific antibody was generated as described previously (Kim et al., J Neurochem 85:1228-1236, 2003). All other chemicals were purchased from Sigma (St. Louis, Mo.).

### Example 1

### EGF Stimulates Insulin Secretion in MIN6 Cells

EGF is produced in the pancreas, has pancreatic effects, and its circulating levels are altered in diabetes (Burgess A W, Br Med Bull 45:401-424, 1989; Kasayama S et al., Proc Natl Acad Sci USA 86:7644-7648, 1989). This example was performed to determine whether EGF could stimulate insulin secretion, and whether insulin secretion by EGF was additive by glucose treatment.

1.1 Method

Cell Culture The mouse insulin-producing cells MIN6m9 provided by Dr. Susumu Seino (Division of Cellular and Molecular Medicine, Kobe University Graduate School of Medicine, Kobe, Japan) were used between passages 19 and 25 and cultured in DMEM containing 25 mM glucose, 10 mM HEPES, 10% (v/v) fetal calf serum, 50 IU/ml penicillin, and 50 μg/ml streptomycin at 37° C. in a humidified CO₂-controlled (5%) incubator. MIN6 cells were transfected using LipofectAMINE, as described previously (Kim et al., J Immunol 163:5462-5470, 1999) Transfection efficiency is about 30-40% by using LipofectAMINE.

Insulin Secretion Assay: Batches of 10-15 isolated islets or 1×10⁶ cells/well grown in 12- or 24-well plates were washed twice with KRB supplemented with 0.2% bovine serum albumin (BSA), and then incubated for 60 min at 37° C. in the KRB solution. We used same number of islets in a same set of experiment. At the end of incubation, the solutions were replaced with fresh KRB containing test reagents and incubated for the designated time. The incubation medium was sampled and centrifuged to remove cells, and the supernatant was assayed for insulin with a radioimmunoassay (RIA) kit (Linco, St. Louis, Mo.).

Statistical Analysis: Results are presented as mean±SE or mean±SD (for PLD activity assay and insulin secretion assay). The statistical significance of differences between means was assessed by Student's t-test. P<0.05 was regarded as statistically significant.

1-2. Insulin Secretion Test of EGF on Mouse MIN6 Insulinoma Cells

To determine whether EGF could stimulate insulin secretion, the present inventors treated mouse MIN6 insulinoma cells with EGF. EGF significantly increased insulin secretion with a 1 min treatment. EGF showed time- and dose-dependent stimulation of insulin secretion from MIN6 cells, with kinetics more rapid than glucose (FIGS. 1A and 1B). Especially 1-2 min and 1.5-15 nM treatment of EGF shows effective time and concentration (FIGS. 1A and 1B).

The MIN6 cells were plated onto 24-well plates and grown for 24 h. The cells were washed twice with KRB supplemented with 0.2% BSA, and then incubated for 60 min at 37° C. in the KRB solution.

In FIG. 1A, at the end of incubation, the solutions were replaced with fresh KRB containing none (NT), 15 nM EGF (human EGF, genbank accession no. CAA34902) or 11 mM glucose, and incubated for 0, 1, 2, 5, or 10 min. The incubation medium was sampled and centrifuged to remove cells, and the supernatant was assayed for insulin levels. The data shown are the mean±S.D. from two independent assays by duplicate. *, P<0.05 compared with not treated (NT) cells.

In FIG. 1B, at the end of incubation, the solutions were replaced with fresh KRB containing 0, 1.5, 15, or 150 nM of EGF, and incubated for 1 min. The incubation medium was sampled and centrifuged to remove cells, and the supernatant was assayed for insulin levels. The data shown are the mean±S.D. from two independent assays by duplicate. *, P<0.05 compared with not treated cells.

1-3. Glucose Treatment on EGF-Induced EGF-Induced Insulin Secretion of Cells

To determine whether insulin secretion by EGF was additive by glucose treatment, the present inventors tested the effect of high (11 mM) glucose on EGF-induced insulin secretion. EGF increased insulin levels at basal concentration of glucose (2.7 mM), and additively increased glucose-induced insulin release at high (11 mM) glucose levels as well (FIG. 1C). Taken together, these data suggest that EGF, like glucose, is an initiator of insulin secretion in pancreatic β-cells and EGF effect on insulin secretion is glucose-independent.

In FIG. 1C, at the end of incubation, the solutions were replaced with fresh KRB containing 0, 15, or 30 nM of EGF in the presence of 2.7 or 11 mM glucose, and incubated for 5 min. The incubation medium was sampled and centrifuged to remove cells, and the supernatant was assayed for insulin levels. The data shown are the mean±S.D. from two independent assays by duplicate. * or **, P<0.05 compared with 2.7 or 11 mM glucose-treated cells.

### Example 2

### EGF-Induced Insulin Secretion is Dependent on Ca²⁺ Influx in MIN6 Cells

Insulin secretion requires increases in intracellular Ca2+ concentrations ([Ca2+]i) (Barg S et al., Diabetes 51 (Suppl 1):S74-82, 2002). This example was carried out to determine the effect of Ca2+ influx on EGF-induced insulin secretion.

2.1 [Ca²⁺]ᵢ Measurement Method

Changes in intracellular Ca2+ levels were monitored using a Ca2+-sensitive dye under a confocal microscope. Cells were loaded with 2 μl Fluo-3 AM for 40 min at room temperature. After washing with Krebs-Ringer bicarbonate (KRB; 129 mM NaCl, 5 mM NaHCO3, 4.8 mM KCl, 1.2 mM KH2PO4, 1.0 mM CaCl2, 1.2 mM MgSO4, 2.7 mM glucose, and 10 mM HEPES, pH 7.4) buffer, the cells were further incubated for 15 min in the absence of Fluo-3 AM to de-esterify the dye. To exclude the possible effects of dye loading, the present inventors normalized levels with saponin at the end of the experiments. To normalize, the present inventors measured the residual fluorescence (Fo) at the end of the experiment, and subtracted that from the fluorescence under experimental conditions (F). Excitation of Fluo-3 AM was performed at 488-nm by an argon laser, and the emission range was 515-nm. Images were captured on an inverted confocal microscope (Zeiss LSM 510 Meta, Oberkochen, Germany) with a 20× objective lens.

2.2. Effect of Ca2+ Influx on EGF-Induced Insulin Secretion

The MIN6 cells were plated onto glass dishes or 24-well plates and grown for 24 h. The cells were washed twice with KRB supplemented with 0.2% BSA, and then incubated for 60 min at 37° C. in the KRB solution.

In FIG. 2A, at the end of incubation, the solutions were replaced with fresh KRB containing Fluo-3 AM dissolved (1 mg/ml) in DMSO, and incubated for 1 h. At the end of incubation, cells were incubated with none (untreated) or EGTA for 30 min and then treated with 15 nM EGF. Images were captured on an inverted confocal microscope with a 20× objective lens. To normalize, the present inventors measured the residual fluorescence (Fo) at the end of the experiment, and subtracted that from the fluorescence under experimental conditions (F). The data shown are the mean±S.E., n=7.

FIG. 2A demonstrated that EGF stimulated extracellular Ca2+ influx, which could be reduced by EGTA treatment. To determine the effect of Ca2+ influx on EGF-induced insulin secretion, the present inventors treated cells with either EGTA to block extracellular Ca2+ influx, or 1,2-Bis(2-aminophenoxy)ethane-N,N,N′,N′-tetraacetic acid tetrakis (acet-oxymethyl ester) (BAPTA/AM) to block both extracellular Ca2+ influx and intracellular Ca2+ release.

In FIG. 2B, at the end of incubation, the solutions were replaced with fresh KRB containing none (untreated), EGTA, or BAPTA/AM and incubated for 30 min, and then treated with 15 nM EGF for 0 or 1 min. The incubation medium was sampled and centrifuged to remove cells, and the supernatant was assayed for insulin levels. The data shown are the mean±S.D. from two independent assays by duplicate. *, P<0.05 compared with EGF-treated cells.

EGF-induced insulin secretion from MIN6 cells was reduced by Ca2+ chelators (FIG. 2B). The same results were also observed in RINm5F cells (data not shown). Taken together, these results suggest that Ca2+ influx is necessary for EGF-induced insulin secretion.

### Example 3

### PLD2 Mediates EGF-Dependent Insulin Secretion in MIN6 Cells

Previous reports suggested that PLD is an important molecule that mediates various exocytosis (Jones D et al., Biochim Biophys Acta 1439:229-244, 1999; Choi W S et al., J Immunol 168:5682-5689, 2002; Metz S A et al., Biochem J 270:427-435, 1990). The present inventors firstly tested the PLD activity in MIN6 cells. PLD was activated rapidly (within 2 min) by EGF stimulation (FIG. 3A). EGF-dependent insulin secretion was inhibited by 1-butanol, a PLD inhibitor, treatment, but not by t-butanol treatment as a control (FIG. 3B). These results suggest that PLD activity is necessary for EGF-induced insulin secretion. The same results were also observed in RINm5F cells (data not shown).

PLD Constructs: The full-length cDNAs of rat PLD1 or human PLD2 were ligated into pcDNA 3.1 vector for transfecting into cells.

SiRNA Sequences: The siRNA of 21-mers corresponding to mouse PLD1 (nucleotides 1099 to 1119, AACACGUUAGCUAAGUGGUAU) (SEQ ID NO:1) or PLD2 sequences (nucleotides 2539 to 2559, AACUCCAUCCAGGCUAUUCUG) (SEQ ID NO:2) were purchased from Dharmacon Research Inc. (Lafayette, Colo.). Results of a BLAST search of all siRNA sequences revealed no significant homology to any other sequences in the database program.

PLD Activity Assay in Cells: Cells grown in 6-well plates were washed twice with KRB, and then labeled with [3H]myristic acid for 4 h at 37° C. in the KRB solution. PLD activity was assayed by measuring the formation of phosphatidylbutanol (PBt) (Kim J H et al., J Immunol 163:5462-5470, 1999). The intensities of PBt spots in the presence of 0.4% 1-butanol were measured, and PLD activity was obtained by subtracting the corresponding intensities of spots obtained in the absence of 1-butanol.

To identify the PLD isozyme responsible for stimulating insulin secretion, the present inventors examined the effect of overexpression and silencing of PLD isozymes. We transfected MIN6 cells with an empty vector, PLD1, or PLD2, and stimulated them with EGF. PLD1 mediated glucose-dependent insulin secretion, as shown previously (data not shown) (Hughes W E et al., J Biol Chem 279:27534-27541, 2004). In contrast, PLD2 exclusively mediated EGF-dependent insulin secretion and overexpression of PLD1 showed a limited effect (upper panel of FIG. 3C). Furthermore, silencing of PLD2, but not PLD1, abolished EGF-induced insulin secretion (upper panel of FIG. 3D). The same results were also observed in RINm5F cells (data not shown). Finally, EGF-dependent PLD activity as measured with PBt formation was modulated exclusively by PLD2 overexpression or silencing (lower panels of FIGS. 3C and 3D), suggesting that PLD2 is required for EGF-stimulated insulin secretion in these cells.

In FIG. 3A, the MIN6 cells were plated onto 6-well plates and grown for 24 h. The cells were washed twice with KRB, and then incubated for 4 h at 37° C. in the KRB solution in the presence of [³H]myristic acid. At the end of incubation, 15 nM EGF stimulation was performed for indicated time. The intensities of PBt spots after 0, 1, 2, 5, or 10 min accumulation in the presence of 1-butanol and EGF were measured, and results were obtained by subtracting the corresponding intensities of spots obtained in the absence of 1-butanol. The data shown are the mean±S.D. from two independent assays by duplicate.

In FIG. 3B, the MIN6 cells were plated onto 24-well plates and grown for 24 h. The cells were washed twice with KRB supplemented with 0.2% BSA, and then incubated for 60 min at 37° C. in the KRB solution. At the end of incubation, the solutions were replaced with fresh KRB containing 0.4% t-butanol or 1-butanol, and incubated for 10 min, and then MIN6 cells were treated with 15 nM EGF for 0 or 1 min. The incubation medium was sampled and centrifuged to remove cells, and the supernatant was assayed for insulin levels. The data shown are the mean±S.D. from two independent assays by duplicate. *, P<0.05 compared with EGF-treated cells.

In FIGS. 3C and 3D, the MIN6 cells were plated onto 24-well plates (for measuring insulin levels) or 6-well plates (for measuring PLD activity) and transfected with the indicated plasmids (vector, PLD1, or PLD2 in FIG. 3C) or siRNAs (control (luciferase), mouse PLD1, or mouse PLD2 in FIG. 3D), grown for 24 h or 72 h. The efficiencies of transfection were approximately 30-40%. For measuring insulin secretion (upper panels), the cells were washed twice with KRB supplemented with 0.2% BSA, and then incubated for 60 min at 37° C. in the KRB solution. At the end of incubation, the solutions were replaced with fresh KRB containing 15 nM EGF for 0 or 1 min. The incubation medium was sampled and centrifuged to remove cells, and the supernatant was assayed for insulin levels. The data shown are the mean±S.D. from two independent assays by duplicate. *, P<0.05 compared with EGF-treated cells. For measuring PLD activity (lower panels), the cells were washed twice with KRB, and then incubated for 4 h at 37° C. in the KRB solution in the presence of [³H]myristic acid. At the end of incubation, 15 nM EGF stimulation was performed for 0 or 1 min. The intensities of PBt spots after 1 min accumulation in the presence of 1-butanol and EGF were measured, and results were obtained by subtracting the corresponding intensities of spots obtained in the absence of 1-butanol. The data shown are the mean±S.D. from two independent assays by duplicate. *, P<0.05 compared with EGF-treated cells. Cells were lysed with KRB containing 0.1% Triton X-100 and subjected to SDS-PAGE and then immunoblotted using anti-PLDs antibody (inner boxes of FIG. 3C and PLD1-inner boxes of FIG. 3D), PLD2-specific antibody (PLD2-inner boxes of FIG. 3D) or actin antibody (actin-inner boxes of FIGS. 3C and 3D).

### Example 4

### PLD Activity is Dependent on Ca²⁺ Influx in MIN6 Cells

Because both Ca²⁺ influx and PLD activity are required for EGF-dependent insulin secretion, this example analyzed the relationship between them by testing the effect of Ca²⁺ influx on PLD activity and the effect of PLD activity on Ca²⁺ influx. Blocking Ca²⁺ influx by using EGTA or BAPTA/AM inhibited most of the PLD activity (FIG. 4A), which correlated with EGF-dependent insulin secretion (FIG. 2B). However, inhibiting PLD activity by silencing PLD isozymes, which the successful silencing of PLDs was confirmed by western blotting, had little effect on EGF-dependent Ca²⁺ influx (FIG. 4B), suggesting that Ca²⁺ influx is an upstream of PLD activation in EGF-dependent insulin secretion.

In FIG. 4A, the MIN6 cells were plated onto 6-well plates and grown for 24 h. The cells were washed twice with KRB, and then incubated for 4 h at 37° C. in the KRB solution in the presence of [³H]myristic acid. At the end of incubation, the solutions were replaced with fresh KRB containing none (untreated), EGTA, or BAPTA/AM and incubated for 30 min, and then treated with 15 nM EGF for 0 or 1 min. The intensities of PBt spots after 1 min accumulation in the presence of 1-butanol were measured, and results were obtained by subtracting the corresponding intensities of spots obtained in the absence of 1-butanol. The data shown are the mean±S.D. from two independent assays by duplicate. *, P<0.05 compared with EGF-treated cells.

In FIG. 4B, the MIN6 cells were plated onto glass dishes and transfected with siRNAs (control (luciferase), mouse PLD1, or mouse PLD2), and then grown for 24 h. The cells were washed twice with KRB supplemented with 0.2% BSA, and then incubated for 60 min at 37° C. in the KRB solution. At the end of incubation, the solutions were replaced with fresh KRB containing Fluo-3 AM dissolved (1 mg/ml) in DMSO and incubated for 1 h. At the end of incubation, cells were treated with 15 nM EGF. Images were captured on an inverted confocal microscope with a 20× objective lens. To normalize, the present inventors measured the residual fluorescence (Fo) at the end of the experiment, and subtracted that from the fluorescence under experimental conditions (F). The data shown are the mean±S.E. of the peak time, n=7. Cells were lysed with KRB containing 0.1% Triton X-100 and subjected to SDS-PAGE and then immunoblotted using anti-PLDs antibody (PLD1-inner box of FIG. 4B), PLD2-specific antibody (PLD2-inner box of FIG. 4B) or actin antibody (actin-inner box of FIG. 4B).

Immunoblot Analysis: Proteins were denatured by boiling for 5 min at 95° C. in Laemmli sample buffer (Laemmli U K et al., Nature 227:680-685, 1970), separated by SDS-PAGE, and immunoblotted, as described previously (Kim J H et al., J Immunol 163:5462-5470, 1999).

### Example 5

### EGF-Stimulated Insulin Secretion in Mouse Pancreatic Islets Requires Ca²⁺ Influx and PLD Activity

To confirm the physiological significance of EGF, Ca²⁺, and PLD on insulin secretion, the present inventors prepared primary cultures of mouse islets. As expected, EGF rapidly increased insulin secretion (FIG. 5A) and the effect was specific among various growth factors (data not shown) with a 1 min treatment. Inhibiting Ca²⁺ influx or PLD activity completely blocked the EGF-induced insulin secretion (FIGS. 5B and 5C), indicating that EGF-stimulated insulin secretion in mouse pancreatic islets requires Ca²⁺ influx and PLD.

To prepare mouse pancreatic islets, pancreatic islets were isolated from 7- to 8-week-old male BALB/c mice (Hyochang Science, Korea), as described previously (Jonas J C, et al., Diabetes 47:1266-1273, 1998). Isolated islets were transferred onto a 12 well plate, with 10-15 islets per well. We used same number of islets in a same set of experiment. The islets were maintained for up to 2 days in RPMI1640 medium containing 5 mM glucose and 10% fetal calf serum, and supplemented with 100 g/ml streptomycin and 100 U/ml penicillin.

The mouse pancreatic islets were plated onto 12-well plates and grown for 24 h. The islets were washed twice with KRB supplemented with 0.2% BSA, and then incubated for 60 min at 37° C. in the KRB solution.

In FIG. 5A, at the end of incubation, the solutions were replaced with fresh KRB and incubated for 1 or 5 min with none (NT), 15 nM EGF, or 11 mM glucose. The incubation medium was sampled and centrifuged to remove islets, and the supernatant was assayed for insulin levels. The data shown are the mean±S.D. from two independent assays by duplicate. * or **, P<0.05 compared with 1 or 5 min-treated islets.

In FIG. 5B, at the end of incubation, the solutions were replaced with fresh KRB containing none (untreated), EGTA or BAPTA/AM, and incubated for 30 min, and then treated with 15 nM EGF for 0 or 1 min. The incubation medium was sampled and centrifuged to remove islets, and the supernatant was assayed for insulin levels. The data shown are the mean±S.D. from two independent assays by duplicate. *, P<0.05 compared with EGF-treated islets.

In FIG. 5C, at the end of incubation, the solutions were replaced with fresh KRB containing 0.4% of t-butanol and 1-butanol, and incubated for 10 min, and then treated with 15 nM EGF for 0 or 1 min. The incubation medium was sampled and centrifuged to remove islets, and the supernatant was assayed for insulin levels. The data shown are the mean±S.D. from two independent assays by duplicate. *, P<0.05 compared with EGF-treated islets.

### Example 6

### EGF Lowers Plasma Glucose and Increases Plasma Insulin Levels

To confirm in vitro findings that EGF could stimulate insulin release, this example characterized the EGF-mediated responses of mouse plasma glucose and insulin levels in normal and obese db/db mice by injecting EGF intravenously.

Measurement of Plasma Glucose and Plasma Insulin Levels: 7- to 8-week-old male ICR mice were purchased from Hyochang Science (Seoul, Korea). C57BLKSJ-db/db mice were purchased from SLC (Japan). After fasting for 6 h, ICR or C57BLKSJ-db/db mice were intravenously injected with saline, EGF, insulin or glucose in the tail vein, and blood samples (0.1 ml) were collected. Concentrations of plasma glucose were measured by the glucose oxidase method with a portable glucose meter (Gluco-Dr, Korea). Plasma was separated by centrifugation and the plasma insulin assay was performed using a RIA kit. Animal care was conducted in accordance with our institution's guidelines.

Measurement of Plasma EGF Levels: 7- to 8-week-old male ICR mice were purchased from Hyochang Science (Seoul, Korea). After fasting for 6 h, ICR mice were orally injected with saline or glucose and blood samples (0.1 ml) were collected in EGTA coated tubes. Concentrations of plasma EGF levels were measured by EGF ELISA kit (KOMA biotech, Korea). Animal care was conducted in accordance with our institution's guidelines.

In preliminary experiments, EGF at 50 g/kg reached a saturated plasma glucose-lowering effect 10 min after the intravenous injection into 7- to 8-week-old male ICR mice (data not shown). This glucose-lowering effect of EGF (50 g/kg) had a similar potency to insulin and had a dose-dependency (FIG. 6A). Moreover, EGF and glucose (0.5 g/kg) also both increased plasma insulin levels (FIG. 6B), suggesting that this glucose lowering effect is due to changes in plasma insulin levels. The kinetics of insulin secretion and changes in glucose were correlated. Furthermore, oral injection of glucose caused the elevation of plasma EGF levels comparing with saline treatment (FIG. 6C). This result suggests that physiological concentration of EGF can be altered by feeding condition and secreted EGF finally regulates insulin secretion. EGF also reduced plasma glucose in obese db/db mice and increased plasma insulin levels (FIGS. 6D and 6E). Taken together, the present inventors conclude that EGF has the ability to stimulate insulin secretion and lowers plasma glucose in normal and diabetic mice model.

In FIG. 6A, 7- to 8-week-old male ICR mice (10 mice/group) received intravenous injections of saline (0.9% NaCl in double distilled water), insulin (0.06 U/kg), or EGF (18.5 or 50 g/kg), and plasma glucose levels were measured. The data shown are the mean±S.E., † (insulin), * (EGF 50 g/kg) or ** (EGF 18.5 g/kg), P<0.05 compared with saline-treated mice in the indicated time.

In FIG. 6B, 7- to 8-week-old male ICR mice (10 mice/group) received intravenous injections of saline, glucose (0.5 g/kg), or EGF (18.5 or 50 g/kg), and plasma insulin levels were measured. The data shown are the mean±S.E., ‡ (glucose), * (EGF 50 g/kg) or ** (EGF 18.5 g/kg), P<0.05 compared with saline-treated mice in the indicated time.

In FIG. 6C, 7- to 8-week-old male ICR mice (10 mice/group) received oral injections of saline, glucose (2 g/kg) and plasma EGF levels were measured. The data shown are the mean±S.E., *, P<0.05 compared with saline-treated mice in the indicated time.

In FIG. 6D, obese C57BLKSJ-db/db mice (6 mice/group) received intravenous injections of saline (0.9% NaCl in double distilled water), insulin (0.06 U/kg), or EGF (50 μg/kg), and plasma glucose levels were measured. The data shown are the mean±S.E., † (insulin) or * (EGF 50 μg/kg), P<0.05 compared with saline-treated mice in the indicated time.

In FIG. 6E, obese C57BLKSJ-db/db mice (6 mice/group) received intravenous injections of saline, glucose (0.5 g/kg), or EGF (50 μg/kg), and plasma insulin levels were measured. Plasma insulin levels before and 10 min after injection were compared. The data shown are the mean±S.E., ‡ (glucose) or * (EGF 50 μg/kg), P<0.05 compared with saline-treated mice in the indicated time. All animals had free access to water. Animal care was conducted in accordance with our institution's guidelines.

While the present invention has been described in detail with reference to the preferred embodiments, those skilled in the art will appreciate that various modifications and substitutions can be made thereto without departing from the spirit and scope of the present invention as set forth in the appended claims.

1. A pharmaceutical composition for preventing or treating diabetes mellitus comprising EGF and a pharmaceutically acceptable carrier.

2. The pharmaceutical composition of claim 1, wherein the EGF stimulates the insulin secretion from pancreatic beta-cell in a glucose-independent manner.

3. The pharmaceutical composition of claim 2, wherein the EGF simulate the insulin secretion through Ca2+ influx and PLP2 activation in pancreatic beta-cells.

4. The pharmaceutical composition of claim 1, wherein the EGF is human EGF.

5. The pharmaceutical composition of claim 1, wherein the EGF is administered in an amount of 5 μg/kg to 100 μg/kg by weight of the subject.

6. The pharmaceutical composition of claim 5, wherein the EGF is administered in an amount of 10 μg/kg to 60 μg/kg by weight of the subject.

7. A method for controlling blood glucose levels in a subject in need thereof comprising administering an effective amount of EGF to the subject.

8. A method for controlling blood insulin levels in a subject in need thereof comprising administering an effective amount of EGF to the subject.

9. The method according to claim 7, where the controlling blood glucose level is performed by regulating the blood insulin levels in a glucose-independent manner.

10. The method according to claim 7, where the effective amount is 5 μg/kg to 100 μg/kg by weight of the subject.

11. The method according to claim 10, where the effective amount is 10 μg/kg to 60 μg/kg by weight of the subject.

12. The method according to claim 8, where the EGF is human EGF.

13. The method according to claim 8, wherein the EGF is administered orally, subcutaneously, intravenously, or intramuscularly.

14. The method according to claim 8, wherein the subject is a patient suffering from diabetes mellitus or a normal subject.

15. The method according to claim 8, wherein the controlling blood glucose level is performed by regulating the blood insulin levels in a glucose-independent manner.

16. The method according to claim 8, wherein the effective amount is 5 μg/kg to 100 μg/kg by weight of the subject.

18. The method according to claim 7, wherein the EGF is human EGF.

19. The method according to claim 7, wherein the EGF is administered orally, subcutaneously, intravenously, or intramuscularly.

20. The method according to claim 7, wherein the subject is a patient suffering from diabetes mellitus or a normal subject.
